(19) United States
(12) Patent Application Publication
(10) Pub. No.: US 2023/0009438 A1
(43) Pub. Date: Jan. 12, 2023

(54) Probiotic formulation for reducing stress, anxiety and depression

(71) Applicant: RATNA SUDHA MADEMPUDI, Shameerpet (IN)

(72) Inventor: RATNA SUDHA MADEMPUDI, Shameerpet (IN)

(21) Appl. No.: 17/370,037

(22) Filed: Jul. 8, 2021

(51) Int. Cl. A61K 35/747; A61K 31/198; A61P 25/20; A61P 25/24; A61P 25/22; A61K 35/742; A61K 35/745

(52) U.S. Cl. CPC A61K 35/747; A61K 31/198; A61P 25/20; A61P 25/24; A61P 25/22; A61K 35/742; A61K 35/745; A61K 2035/115

(57) ABSTRACT

Stress is any demand placed on the brain or physical body, thereby leading to anxiety and depression and it is a common phenomenon experienced by humans and animals. Stress, anxiety and depression often leads to serious mental ill health. People with anxiety disorders respond to certain things or situations with fear and dread and display physical signs such as a pounding heart and sweating. Often, people lose their willpower, concentration and interest in day to day activities, are drained of energy and can even become devoid of any emotion.

It is documented that low activity of certain neurotransmitters can result in anxiety, chronic stress, depression, difficulty in concentrating, memory problems, muscle pain, headaches, insomnia and sleep problems. Therefore, if the level of these neurotransmitters is raised, the problem of stress, anxiety and depression can be cured. The invention describes a formulation to naturally increase the levels of certain neurotransmitters in the body, thereby reducing stress, anxiety and depression.

### TECHNICAL FIELD

The present subject matter generally relates to a probiotic formulation, more particularly the subject matter relates to a probiotic formulation for relieving stress and anxiety and even more particularly, the subject matter relates to a probiotic formulation for relieving stress and anxiety using probiotic microorganisms.

### BACKGROUND

Stress is a common phenomenon experienced by humans and animals. Stress is any demand placed on the brain or physical body, thereby leading to anxiety and depression. The feeling of being stressed makes one feel frustrated or nervous.

Stress, anxiety and depression often leads to serious mental ill health. If not addressed properly, stress can lead to an anxiety disorder, which is a serious mental illness. People with anxiety disorders respond to certain things or situations with fear and dread and display physical signs such as a pounding heart and sweating. Anxiety includes panic disorders, social anxiety disorder, specific phobias, generalized anxiety disorder and post-traumatic stress disorder and can get worse if not treated. Depression is a feeling of hopelessness where a person is sure that the future will turn out to be bad. Often, people lose their willpower, concentration and interest in day to day activities, are drained of energy and can even become devoid of any emotion. Severe depression makes a person sad, angry and irritable and often has a feeling of guilt and self-loathing.

Though stress, anxiety and depression are very complex phenomena and their causes cannot be explained easily, recent studies have shown that the reason of anxiety and depression may be linked to neurotransmitters, or due to presence or absence of different chemical compounds related to the endocrine, and immune systems, such as serotonin, thyroid hormone, epinephrine/norepinephrine, endorphins, dopamine and the neuro transmitter gamma amino butyric acid (GABA).

Various methods are available for treatment of stress, anxiety and depression. U.S. Pat. No. 5,681,578 discloses a composition for relieving stress anxiety, grief, and depression. The composition includes GABA (gamma amino butyric acid), glutamine, glycine, magnesium, passionflower, primula officinalis, and vitamin B-6. Chinese patent CN102228255 describes nutritional medicinal granules capable of relieving mental pressure and resisting depression. The medicinal granules include nutritional oils, plantain powder, fruit extract, fish oil, tryptophan, tyrosine, folic acid and other natural ingredients. The granules disclosed by the invention have good mouthfeel and have favorable action on improving depressed people with irritability, great mood fluctuations, obvious energy reduction, causeless continuous fatigue, insomnia, early sleep, hypersomnia, and concentration difficulty or plummet. Korean patent KR20140115079 reveals novel strains of Lactobacillus plantarum K255 with high productivity of gamma-aminobutyric acid. The strain also has bile resistance and strong acid resistance and can be used as a fermentation starter strain for producing fermented milk or soybean milk. Chinese patent CN106434482 describes Lactobacillus plantarum SG5 for producing gamma-aminobutyric acid. The strain is also used for fermentation, and the benefits include lowering blood pressure, promoting growth hormone secretion, lowering cholesterol and provide basis for the development of new GABA-rich health-care food. Japanese patent JP2001120179 describes a method for production of GABA-containing fermented milk using lactic bacteria and/or bacteria belonging to the genus Bifidobacterium having glutamic acid decarboxylase. PCT application WO 2013/107913 describes a GABA-producing culturable bacteria derived from the human gastrointestinal tract.

It is documented that low GABA activity in the body can result in anxiety, chronic stress, depression, difficulty in concentrating, memory problems, muscle pain, headaches, insomnia and sleep problems. Therefore, if the level of GABA is increased to a required level, the problem of stress, anxiety and depression can be cured.

GABA supplements, if taken directly may cause side effects such as depressed mood, drowsiness, nausea, etc. Accordingly, there is a need to develop a natural and safe composition having no side effects, for effectively increasing the level of gamma amino butyric acid (GABA) in the body.

### OBJECTS

Accordingly, the main object of the present invention is to provide a formulation to naturally increase the level of gamma amino butyric acid (GABA) in the body thereby helping to reduce stress, anxiety and depression.

### SUMMARY OF THE INVENTION

Probiotic microbes are recognized for their role in producing neurotransmitters such as gamma-aminobutyric acid (GABA), and serotonin. Proper levels of these neurotransmitters decrease the stress of an individual. Also, deficiency of these neurotransmitters causes high level of stress and anxiety and related disorders such as high blood pressure in individuals. High levels of these neurotransmitters may cause side effects such as depressed mood, drowsiness, nausea, etc. Therefore, proper level of neurotransmitters is necessary. Probiotic microbes help to maintain proper level of neurotransmitters and results in beneficial effect on the individual.

In accordance with this invention, the probiotic microbes in the present invention, specifically produce enzymes for increasing the level of GABA in the body. GABA has also been detected in other peripheral tissues including intestines, stomach, fallopian tubes, uterus, ovaries, testes, kidneys, urinary bladder, the lungs and liver. Besides the nervous system, GABA also affects the production of insulin. Therefore, increasing the level of GABA in the body may be of special help in persons with diabetes.

The formulation comprises probiotic microorganisms. Probiotics are non-pathogenic and beneficial microbes taken orally in form of tablets, powders in sachets or liquids. They are introduced in an individual in form of spores or vegetative cells. The microbes lodge themselves in the gut, colonize the gut environment and breakdown the food entering in the intestine, thereby aiding in proper digestion of food in the individual. They also break down the food in the gut to produce useful metabolites.

The present invention describes a probiotic formulation for lowering stress and anxiety. The formulation contains a consortium of probiotic microbes, belonging to Bacillus sp., Lactobacillus sp. and Bifidobacterium sp. Specifically, the consortium is of probiotic Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01. All the microbes are non-pathogenic to humans and animals. The cultures are deposited in the MTCC culture collection.

The effective amount of probiotic Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01 is 10⁶ colony forming units to 10¹² colony forming units per dosage of said formulation, but preferably 10⁸ colony forming units to 10¹⁰ colony forming units per dosage of said formulation. The probiotic Bacillus sp. and Lactobacillus sp. are in form of viable spores or in form of vegetative cells. The microbes are capable of germination and growth in the gut of the individual.

The formulation also contains a non-essential amino acid glutamine. Non-essential amino acids are generally synthesized by the body itself and are not required to be given externally. In the present formulation, the non-essential amino acid is given as a precursor to the microbes so that they can utilize the amino acid and produce a useful metabolite. At least one or all of the said microbes is capable of degrading the said non-essential amino acid. The amount of non-essential amino acid is 100 mg to 500 mg, preferably 200 mg to 300 mg per single dose of the formulation.

The invention describes a method of producing an inhibitory neurotransmitter in an individual. The method includes the steps of introducing a probiotic formulation into the gut of the said individual, by oral route. The formulation is made of a consortium of microbes and a non-essential amino acid and the consortium of microbes metabolizes the non-essential amino acid to produce a metabolite which is a neurotransmitter. This neurotransmitter is transported to the brain and results in lowering stress and anxiety of an individual.

The probiotic formulation described in the invention is introduced into the gut of an individual by oral route in form of tablets, capsules powders or liquids. The microbes are resistant to the environment of the stomach and the small intestine, and hence can move through this environment and lodge themselves in the gut of the individual.

The non-essential amino acid added to the formulation is glutamine. It is understood here, that glutamine is the precursor to the formation of Gama Amino Butyric Acid (GABA) in the brain. Also, the consortium of microbes in the probiotic formulation metabolize the glutamine to produce the inhibitory neurotransmitter is GABA.

The invention describes a method of reducing stress, anxiety and depression in an individual. The method includes the steps of administering an effective dose of a probiotic formulation into the individual. The probiotic formulation contains microbes and the microbes lodge themselves in the gut of an individual and produce an inhibitory neurotransmitter and the said neurotransmitter reduces stress, anxiety and depression in the individual. The gut and the brain are connected through the gut brain axis.

### DETAILED DESCRIPTION OF THE INVENTION

In accordance with this invention, a probiotic formulation is described.

According to one object of the present invention, a formulation for lowering stress and anxiety is described and the formulation comprises probiotic microorganisms.

In an object, the formulation contains a consortium of probiotic microbes, belonging to Bacillus sp., Lactobacillus sp. and Bifidobacterium sp. Specifically, the consortium is of probiotic Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01.

In another object, the microbial consortium has beneficial microbes and these microbes are non-pathogenic to humans and animals.

In one more object, the formulation is introduced into an individual, orally in form of tablets, capsules, powders or liquid.

In one more object, the microbes are introduced into the gut of an individual in form of spores or vegetative cells, and the microbes lodge themselves in the gut, grow and colonize the gut environment and breakdown the food entering in the intestine, thereby aiding in proper digestion of food in the individual and break down the food in the gut to produce useful metabolites.

In yet another object, the effective amount of probiotic Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01 is 10⁶ colony forming units to 10¹² colony forming units per dosage of the said formulation, but preferably 10⁸ colony forming units to 10¹⁰ colony forming units per dosage of the said formulation.

In an object, the probiotic formulation contains 100 mg to 500 mg, preferably 200 mg to 300 mg of a non-essential amino acid 190 glutamine and the glutamine is given as a precursor to the microbial consortium and at least one or all of the said microbes is capable of degrading glutamine.

In another object, the invention describes a method of producing an inhibitory neurotransmitter in an individual. The method includes the steps of introducing a probiotic formulation into the gut of the said individual, by oral route. The formulation is made of a consortium of microbes and a non-essential amino acid and the consortium of microbes metabolizes the non-essential amino acid to produce a metabolite which is a neurotransmitter. This neurotransmitter is transported to the brain and results in lowering stress and anxiety of an individual.

In yet another object, the probiotic formulation is introduced into the gut of an individual by oral route in form of tablets, capsules powders or liquids and the microbes are resistant to the environment of the stomach and the small intestine, and hence can move through this environment and lodge themselves in the gut of the individual.

In one more object, the non-essential amino acid added to the formulation is glutamine and the consortium of microbes in the probiotic formulation metabolize the glutamine to produce the inhibitory neurotransmitter is Gama Amino Butyric Acid (GABA).

In yet another object, a method of reducing stress, anxiety and depression in an individual is described, and the method includes the steps of administering an effective dose of a probiotic formulation into the individual. The probiotic formulation contains microbes and the microbes lodge themselves in the gut of an individual and produce an inhibitory neurotransmitter and the said neurotransmitter reduces stress, anxiety and depression in the individual.

### DETAILED DESCRIPTION OF THE SUBJECT MATTER WITH RESPECT TO EXAMPLES

Before the present subject matter is described in further detail, it is to be understood that the subject matter is not limited to the particular embodiments described, and as such may, of course, vary. It is also to be understood that the terminology used herein is for the purpose of describing particular embodiments only, and is not intended to be limiting, since the scope of the present invention will be limited only by the appended claims. It must be noted that as used herein and in the appended claims, the singular forms “a”, “an”, and “the” do not denote a limitation of quantity, but rather denote the presence of at least one of the referenced items unless the context clearly dictates otherwise.

### Example 1

A study was conducted on students taking their undergraduate exam. The trial was double blind, and participants were randomized and distributed equally into placebo and treatment group. The ethical guidelines of Indian Council of Medical Research (ICMR) on biomedical research on human subject, the principles of the Declaration of Helsinki and ICH-GCP code of conduct for research involving human volunteers were followed. The informed consent was obtained from participants prior to study initiation.

Total 80 students were enrolled, out of 80, 74 were able to complete the study schedule. Out of 74 students, 36 received the multi-strain probiotic capsule with glutamine and 38 received placebo capsule (microcrystalline cellulose) 2 times a day.

The mean age of the students was 21.4±1.5 years (range 18-24 years) and weight 55.72±7.12 kg. Number of female students was 63 (78.75%) and 17 (21.25%) were male. All students participated in this study were of Indian origin and on vegan diet, which mainly consist of rice, Indian whole-wheat chapati, beans and vegetable curry etc.

Inclusion criteria: Students of age between 18 to 24 years facing examination stress; no reported cardiac, renal or hepatic conditions; free from psychiatric conditions other than stress; having no allergies or intolerance to lactose or gluten; on no medications, alcohol or drugs; female students not on hormonal contraceptives. Exclusion criteria: suffering from any chronic physical, hormonal or psychiatric illness; using hormonal birth-control measures; currently on any medication on a regular basis; using health supplements like multivitamins or minerals; pregnant or lactating; had a substance dependency; enrolled in any clinical study within the last 6 months; on any antibiotic usage or had consumed probiotics three weeks prior to the study.

The students complying with inclusion/exclusion criteria and signed informed consent form were interviewed by psychiatrist for examination stress to facilitate enrolments. All 80 eligible students were randomized 1:1 ratio by simple randomization (tossing the coin) into two arms i.e. probiotic and placebo. The information of randomization was kept blind in identical, opaque sealed envelopes. The investigators who were blinded to the treatment, assigned intervention to the students based on randomization numbers. Besides this, both probiotic and placebo capsules are identical in appearance, except the presence of active ingredients.

The students were instructed to take probiotic or placebo capsules twice a day for the period of 28 days (2 weeks before the examinations commenced and during the examination). They were provided a blinded kit containing capsules in a quantity adequate for the entire duration of the study. To verify treatment compliance, a follow-up telephone call was made weekly. To assess the stress, a validated questionnaire such as, Perceived Stress Scale (PSS), Depression Anxiety Stress Scale (DASS) and State—Trait Anxiety Inventory (STAI) were provided. Serum cortisol levels were determined at the baseline and end of the treatment (EOT).

Efficacy Parameters

Perceived Stress Scale (PSS): PSS is the most widely used psychological instrument for measuring the perception of stress. The PSS consists of ten questions, each measures the degree to which situations in one's life are appraised as stressful. For each question, the score ranges from 0 to 4 (0=never, 1=almost never, 2=sometimes, 3=fairly often and 4=very often). The score ranges from 0 to 40, whereas, zero reflect virtual absence of stress in one's life, 0-13 indicate low stress, 4-26 moderate stress and 27-40 high stress.

Depression Anxiety Stress Scale (DASS): DASS-21 items are a set of three self-report scales designed to measure the emotional states of depression, anxiety and stress. The score of 0-14 indicates normal, 15-18 mild, 19-25 moderate, 26-33 severe and 34+ plus indicates extremely severe stress.

State—Trait Anxiety Inventory (STAI): STAI is a commonly used measure of trait and state anxiety. It can be used to diagnose anxiety and to distinguish it from depressive syndromes. Form with 20 items for state anxiety was used in the study. Each question had a range from the score of 1-4 with increased scores indicating greater stress.

Serum Cortisol Levels: The early morning fasting blood samples of all participants were collected at the baseline and end of the treatment by routine procedure. The serum cortisol levels were analyzed.

Statistical Analysis: The study is sufficiently powered for 74 participants, keeping margin of error 5% (0.05), power of the study 80% and confidence interval of 95%. A t-test was used to assess the statistical significance. P value less than 0.05 was considered as significant. All efficacy analyses were performed on per-protocol population by using GraphPad Prism (USA).

Results

Perceived Stress Scale (PSS): In probiotic group, the mean PSS score significantly decreased from the baseline (24.44±2.13) to the end of the treatment (EOT: day 28; 15.00±2.18), whereas in placebo it was significantly increased from 22.39±2.63 to 23.84±3.32. The percent change from baseline to EOT in probiotic group was −38.62% and placebo 6.47%. Moreover, at the end of the trial, multi-strain probiotic supplement significantly reduced PSS score as compared with placebo.

Depression Anxiety Stress Scale (DASS): The change in mean DASS score recorded for probiotic group was significant as compared with placebo. Baseline score (20.83±3.89) in probiotic group was significantly decreased (12.72±4.46) at EOT, however, in placebo it significantly increased from 21.47±4.52 to 23.79±4.43. In probiotic group, −38.93% change was estimated from baseline to EOT, whereas in placebo it was 10.80%.

State—Trait Anxiety Inventory (STAI): At EOT, the mean baseline score (48.58±8.25) of STAI was significantly decreased (39.78±7.61) in probiotic group, while in placebo it significantly increased from 45.61±6.82 to 49.89±7.02. The % baseline change was −18.11% in probiotic group and 9.38% in placebo. Moreover, STAI score was significantly decreased as compared with placebo.

Serum Cortisol Levels: The serum cortisol levels in probiotic group were significantly decreased as compared to placebo. The significant reduction in baseline cortisol levels (23.29±2.99 μg/dl) were estimated at EOT (13.27±2.67 μg/dl) in probiotic group. However, placebo showed significantly increased levels of cortisol from baseline (20.06±2.42 μg/dl) to EOT (22.03±2.72 μg/dl). A −43.02% change was recorded from baseline to EOT in probiotic group, while placebo showed 9.82% change.

The results show that the daily (twice-a-day) supplementation of multi-strain probiotic with glutamine for 28 days significantly reduced level of stress in students facing examination. The stress was assessed by using most widely used psychological instruments such as perceived stress scale (PSS), depression anxiety stress scale (DASS), state—trait anxiety inventory (STAI) and serum cortisol levels. Furthermore, no adverse events (AE's) were reported indicating the safety of the probiotic formulation.

While the invention has been described with reference to preferred embodiments, it will be understood by those skilled in the art that various changes may be made, and equivalents may be substituted for elements thereof without departing from the scope of the invention. In addition, the description is not intended to limit the scope of the invention to the particular forms set forth, but on the contrary, it is intended to cover such alternatives, modifications, and equivalents as may be included within the spirit and scope of the invention as indicated by the following appended claims.

I claim:

1. A probiotic formulation for lowering stress, anxiety and depression, said formulation comprising at least a non-essential amino acid and a consortium of probiotic microbes, said consortium further comprising Bacillus sp., Lactobacillus sp. and Bifidobacterium sp. wherein, the said microbes are non-pathogenic to humans and animals and wherein at least one of the said microbes is capable of degrading the said non-essential amino acid to produce an inhibitory neurotransmitter and the said neurotransmitter lowers stress, anxiety and depression in an individual.

2. The probiotic formulation of claim 1, wherein the non-essential amino acid is glutamine.

3. The probiotic formulation of claim 1, wherein the consortium of microbes comprises probiotic Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01.

4. The probiotic formulation of claim 3, wherein the probiotic Bacillus coagulans Unique IS2 sporulates and the spores are capable of germination and growth in the gut of the individual.

5. The probiotic formulation of claim 1, wherein the probiotic Lactobacillus rhamnosus UBLR58, Bifidobacterium lactis UBBLa70, Lactobacillus plantarum UBLP40, Bifidobacterium breve UBBr01 and Bifidobacterium infantis UBBI01 are vegetative cells and the vegetative cells are capable of germination and growth in the gut of the individual.

6. The probiotic formulation of claim 3, wherein the effective amount of probiotic Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01 is 10⁶ colony forming units to 10¹² colony forming units per dosage of said formulation, but preferably 10⁸ colony forming units to 10¹⁰ colony forming units per gram per dosage of said formulation.

7. The probiotic formulation of claim 2, wherein the amount of non-essential amino acid is 100 mg to 500 mg, preferably 200 mg to 300 mg per dosage of said formulation.

8. A method of producing an inhibitory neurotransmitter in an individual, said method comprising introducing a probiotic formulation into the gut of the said individual wherein said probiotic formulation comprises at least a non-essential amino acid and a consortium of microbes wherein said consortium of microbes further comprises Bacillus coagulans Unique IS2, Lactobacillus rhamnosus UBLR58, Bifidobacterium lactis UBBLa70, Lactobacillus plantarum UBLP40, Bifidobacterium breve UBBr01 and Bifidobacterium infantis UBBI01 and wherein the consortium of microbes metabolizes the non-essential amino acid to produce the inhibitory neurotransmitter.

9. The method of claim 8, wherein the probiotic formulation is introduced into the gut of an individual by oral route and wherein the consortium of microbes in the said formulation lodge themselves in the gut of the individual.

10. The method of claim 8, wherein non-essential amino acid is glutamine and the consortium of microbes in the probiotic formulation metabolize the glutamine to produce an inhibitory neurotransmitter is Gama Amino Butyric Acid (GABA).

11. The probiotic formulation of claim 1, wherein the said formulation is in form of powder in sachets, tablets, capsules or liquids.

12. A method of reducing stress, anxiety and depression in an individual, said method comprising administering an effective dose of a probiotic formulation into the individual wherein the said formulation aids in producing an inhibitory neurotransmitter and the said neurotransmitter reduces stress, anxiety and depression in the individual.
